(19) United States
(12) Patent Application Publication
(10) Pub. No.: US 2013/0156733 A1
(43) Pub. Date: Jun. 20, 2013

(54) INFLUENZA VIRUS POPULATIONS, METHODS OF USE AND METHODS OF MAKING THEREOF

(71) Applicants: Philip I. Marcus, Storrs, CT (US); John M. Ngunjiri, Willimantic, CT (US); Margaret J. Sekellick, Storrs, CT (US); Chang-Won Lee, Wooster, OH (US)

(72) Inventors: Philip I. Marcus, Storrs, CT (US); John M. Ngunjiri, Willimantic, CT (US); Margaret J. Sekellick, Storrs, CT (US); Chang-Won Lee, Wooster, OH (US)

(21) Appl. No.: 13/721,787

(22) Filed: Dec. 20, 2012

Related U.S. Application Data
(60) Provisional application No. 61/577,889, filed on Dec. 20, 2011.

(51) Int. Cl. A61K 35/76; A61K 38/21

(52) U.S. Cl. CPC A61K 35/76; A61K 38/21
USPC 424/85.4; 424/93.6; 435/5

(57) ABSTRACT

Disclosed herein are live attenuated influenza virus compositions for inducing interferon comprising quantified subpopulations of interferon-inducing particles and defective-interfering particles. The live attenuated influenza virus compositions are particularly useful to induce interferon in an individual having or suspected of having a viral infection. Further, by infecting mammalian and avian cells with the live attenuated influenza virus compositions, the compositions optimized for mammalian or avian species can be selected based on the ratio of defective-interfering particles to interferon-inducing particles.

### CROSS-REFERENCE TO RELATED APPLICATIONS

This application claims priority to U.S. Provisional Application Ser. No. 61/577,889 filed on Dec. 20, 2011, which is incorporated herein by reference in its entirety.

### FIELD OF THE DISCLOSURE

Described herein are broad spectrum attenuated and/or inactivated influenza virus preparations containing optimized ratios of defective-interfering particles to interferon-inducing particles which are particularly useful to enhance the induction of interferon which acts as a potent natural adjuvant and a natural antiviral agent.

### BACKGROUND

Interferons (IFNs) are proteins made and released by host cells in response to the presence of pathogens including viruses and bacteria. IFNs allow communication between cells to trigger the protective defenses of the immune system that eradicate pathogens or tumors. The immune effects of interferon can be used to treat viral infections or cancer, for example, however, injection of exogenously produced interferon is often not effective and the effective doses are often associated with detrimental side effects. Efforts have also focused on approaches to stimulate the body to make IFN. Poly(rI):poly(rC) has been used as a reasonably good inducer of interferon in vitro and in vivo. However, this dsRNA tends to be toxic to cells and the effective dose is close to the pharmacologically toxic dose. Derivatives of poly(rI):poly(rC) were synthesized that were less toxic because they were destroyed more readily by serum nucleases.

What is needed are alternative approaches to produce optimal amounts of IFNs that are localized to the site of the body where treatment is directed rather than the untargeted injection or production of IFN that causes systemic side effects.

### SUMMARY

In one aspect, a live attenuated influenza virus composition for inducing interferon, comprises

a subpopulation of interferon inducing particles that contain a delNS1 gene, and

a subpopulation of defective-interfering particles,

wherein the ratio of defective-interfering particles to interferon-inducing particles is greater than 10:1,

wherein the subpopulation of interferon-inducing particles, the subpopulation of defective-interfering particles, or both, is UV irradiated at 254 ±20 nm, at 500 to 2,500 ergs/mm², and

wherein the live attenuated influenza virus composition has hyper interferon-inducing capacity which exceeds by 10 fold or higher the basal levels induced by wild type influenza viruses.

In another aspect, a method of inducing interferon in an individual having or suspected of having a viral infection, comprises administering to the individual a live attenuated influenza virus composition comprising

a subpopulation of interferon inducing particles that contain a delNS1 gene, or a subpopulation of interferon inducing particles that contain a full size NS1 gene, and

a subpopulation of defective-interfering particles,

wherein the ratio of defective-interfering particles to interferon-inducing particles is greater than 10, and

wherein the subpopulation of interferon-inducing particles, the subpopulation of defective-interfering particles, or both, is UV irradiated at 254±20 nm and at 500 to 2,500 ergs/mm².

In a still further aspect, a method of screening an influenza virus preparation for hyper interferon-inducing capacity which exceeds by 10 fold or higher the basal levels induced by wild type influenza viruses, comprises

preparing an influenza virus preparation from an influenza virus with a delNS1 gene or a full-size NS1 gene that expresses an NS1 protein lacking the capacity to suppress interferon induction, wherein the preparation contains interferon-inducing particles and defective-interfering particles, and

quantifying the ratio of defective-interfering particles to interferon-inducing particles in mammalian cells, and

determining that the influenza virus preparation is suitable for treating mammalian species when the ratio of defective-interfering particles to interferon-inducing particles in the influenza virus preparation is greater than 10:1 in mammalian cells,

or

quantifying the ratio of defective-interfering particles to interferon-inducing particles in avian cells, and

determining that the influenza virus preparation is suitable for treating avian species when the ratio of defective-interfering particles to interferon-inducing particles in the influenza virus preparation is greater than 10 in avian cells.

### BRIEF DESCRIPTION OF THE DRAWINGS

FIGS. 1, 2, and 3 show interferon (IFN) induction dose-response curves in aged chick embryo cells (FIG. 1), monkey Marc-145 cells (FIG. 2), and mouse L(Y) cells (FIG. 3). The ordinates represent the yield of IFN following exposure of cells to increasing multiplicities of plaque-forming (infectious) particles (lower abscissae). The upper abscissae represent calculated multiplicities of IFN-inducing particles (IFPs). The plots in FIGS. 2 and 3 are arranged in the order of decreasing QYs of IFN: FIG. 2, D-del-pc2 (A)>D-del-pc1 (B)>D-del-pc3 (C)>>>D-del-pc4 (D)>>>Wild type (E); FIG. 3, D-del-pc2 (A)>>>D-del-pc1 (B)>D-del-pc3 (C)>>>D-del-pc4 (D)≈Wild type (E). Different symbols in A to E represent data points from independent experiments. The dotted lines represent the expected types of IFN induction curves following the Poisson distribution, P(r)=(e^{-m}m^{r})/r!, of IFPs among the cell population. Type r=1 curves (FIG. 2A, B; FIG. 3A, B) best fit a model in which cells that receive 1 and only 1 IFP respond by producing a QY of IFN, whereas cells that receive 2 or more IFPs produce little or no IFN, i.e., P₍ᵣ₌₁₎=(em/e^{m})QY. Type r=2 curves (FIG. 1C, D; FIG. 2C, D; FIG. 3A to D) best fit a model in which cells that receive 2 and only 2 IFPs respond by producing a QY of IFN, whereas cells that receive 1 or more than 2 IFPs produce little or no IFN, i.e., P₍ᵣ₌₂₎=(e²m²/4e^{m})QY. Type r≧1 curves (FIG. 1A, B) best fit a class of cells in which a full yield of IFN is induced in any cell that receives 1 or more IFPs, i.e., P_{(r≧1)}=(1−e^{-m})QY. Where P(r) is the fraction of cells that get r IFPs when exposed to a multiplicity of m, r is the actual number of IFPs that enter the cell, r! is the factorial of r, m is the multiplicity of IFPs (i.e., IFP/cell ratio), and e is the base of the natural logarithm (e=2.71828 . . . ).

FIGS. 4, 5, and 6 show the dynamics of infection simulations in monkey Marc-145 cells (FIG. 4), mouse L(Y) cells (FIG. 5), and chicken CEC (FIG. 6).

FIG. 7 shows RT-PCR detection of polymerase gene segment-derived subgenomic defective-interfering RNAs.

FIGS. 8 and 9 show the effect of the SLSYSINWRH (SEQ ID NO. 1) amino acid motif at the C-terminus of NS1 protein of variant D-del-pc2 on interferon inducing capacity in mouse L(Y) cells (FIG. 8) and monkey Marc-145 cells (FIG. 9).

FIG. 10 shows in vivo establishment of naturally selected D-del-pc2-NcM as effective M-LAIV/H-IFPs in mice.

FIG. 11 shows the effect of UV (254 nm) radiation on interferon inducing capacity of M-LAIV/H-IFPs candidates.

### DETAILED DESCRIPTION

Currently, Live-Attenuated Influenza Vaccines (LAIVs) are designed to elicit antibodies directed primarily toward single or heterologous hemagglutinin (HA) antigens. A robust induction of IFN by the LAIVs correlates with a stronger stimulation of antibody response and cell-mediated immunity because IFN is a natural adjuvant. Following treatment with LAIVs, the host endogenously produces different types of IFNs (cytokines) some of which can act synergistically to elicit a greater antiviral effect. IFN also acts as the first line of defense against virus infection: its induction/production occurs within hours after infection, and many days before antibodies appear as a result of the adaptive immune response. It is now known that LAIVs contain subpopulations of virus particles, such as interferon-inducing particles (IFPs) and defective-interfering particles (DIPs). However, tailored modulation of IFN induction by these virus subpopulations has not been attempted or refined. Previous reports that point out the importance of NS1 proteins in IFN induction did not recognize that only a fraction of the particles which expressed C-terminally truncated NS1 proteins were capable of inducing IFN. Quantitative analysis currently shows that only a small fraction of influenza virus particles that express C-terminally truncated NS1 proteins are capable of functioning as IFPs. Previous reports do not recognize the importance of IFPs nor attempt to augment the production of IFN through the manipulation of subpopulations of particles.

Described herein is essentially a modified LAIV (M-LAIV) that, through the action of hyperactive IFPs (H-IFPs), induces robust amounts of IFN, which can be 10 fold or higher to those induced by wild type influenza viruses such as, but not limited to, A/PR/8/34 (H1N1) or A/TK/OR/71 (H7N3). In turn, IFN signals cells to develop an antiviral state against a broad spectrum of viruses—including, but not limited to, influenza viruses. The hyper levels of IFN induced endogenously by the compositions described herein also serve as a natural immunoadjuvant to boost the adaptive immune system. M-LAIV/H-IFPs could be used in any situation where a large quantity of endogenously-induced IFN might be advantageous; for example, therapy designed to control IFN-sensitive viruses, hepatitis B or C, multiple sclerosis, or certain cancers. Thus, the administration of a single dose, preferably intranasal, of M-LAIV/H-IFPs would suffice to stimulate the host to produce large amounts of appropriate mixtures of IFNs naturally, which would in turn act to ameliorate or treat viral and immune-based disorders.

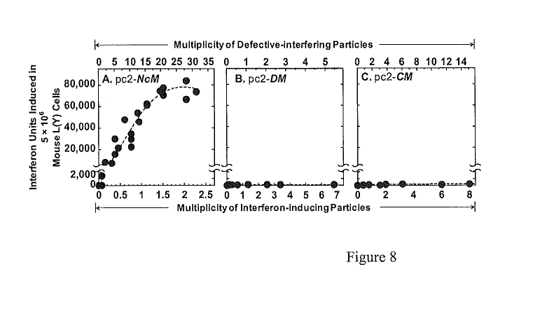

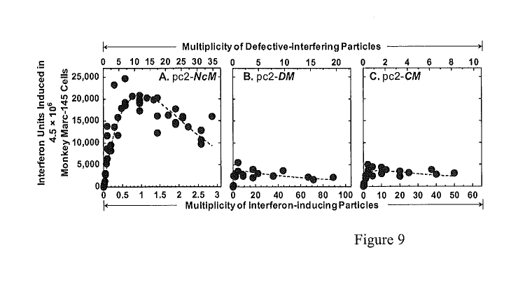

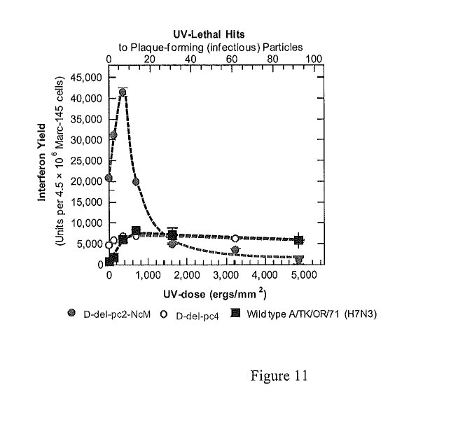

LAIVs are designed to elicit antibodies directed primarily toward single or heterologous hemagglutinin (HA) antigens, and it is well recognized that a robust endogenous induction of IFN enhances this antibody response. However, prior reports are not based on defined modulation of the virus subpopulations of IFPs that are absolutely responsible for this response. It has not been suggested previously to use IFPs specifically to induce IFN, or to augment the production of IFN through the addition of subpopulations like DIPs or UV-irradiated IFPs to increase the efficiency of IFPs to achieve a more robust induction of IFN (see FIG. 8; FIG. 9; FIG. 11).

The influenza A virus genome contains eight segments of single-stranded RNA of negative polarity, coding for eleven functional proteins [PB1, PB1-F2, PB2, PA, HA, NP, NA, M1, M2, NS2/NEP, and NS1 proteins]. The NS1 (nonstructural protein 1) is abundant in influenza virus infected cells, but has not been detected in virions. It is found in the nucleus and also in the cytoplasm during the replication cycle of influenza virus. Studies with temperature-sensitive (ts) influenza mutants carrying lesions in the NS (nonstructural) gene segment suggested that the NS1 protein is a transcriptional and post-transcriptional regulator of mechanisms by which the virus is able to inhibit host cell gene expression and to stimulate viral protein synthesis. Like many other proteins that regulate post-transcriptional processes, the NS1 protein interacts with specific RNA sequences and structures.

Described herein are live attenuated influenza virus compositions comprising a subpopulation of IFPs and a subpopulation of DIPs. In specific embodiments, the subpopulations of IFPs contain delNS1 genes.

IFPs constitute the source of the IFN-inducing capacity of the virus population. They are generated naturally in the course of replication of the M-LAIV/H-IFPs variants and are usually present in numbers that exceed the infectious particle titer. IFPs need not be infectious to induce IFN.

In certain embodiments, the IFPs have full-size NS1 genes that express NS1 proteins that do not have the capacity to suppress interferon induction, thereby functioning as hyper-interferon-inducing IFPs.

In other embodiments, the IFPs have delNS1 genes. As used herein, an influenza virus with a delNS1 gene is a designed deletion mutant of NS1, in which only a part, and not the entire gene, is deleted. U.S. Patent Publication No. 2009/0053264 which describes NS1 deletion is incorporated herein by reference for its description of NS1 and deletions of NS1. Deletion of the entire NS1 gene would be referred to as an NS1 knockout. NS1 deletion mutants were found to be attenuated in vivo, and to induce high yields of IFN. Vaccination with NS1 deletion mutants blocked replication and spread of a wild-type challenge virus, and afforded protection from disease.

More specifically, an influenza virus with a delNS1 gene encodes an NS1 protein having truncated C-terminus, wherein the truncated C-terminus includes sequences of 2-20 nonconsensus amino acid residues, for example, wherein nonconsensus amino acid residues are residues that are not present in the wild type, full-length NS1 protein sequence. It is shown that the nonconsensus amino acids convert a weak inducer of interferon to a hyper-inducer of interferon (FIGS. 8 and 9). The influenza virus can be an avian or mammalian influenza virus. In one embodiment, the influenza virus is an avian influenza virus. The wild type avian NS1 sequence is a 230 amino acid sequence (SEQ ID NO. 2) (GenBank: AAB93939.1).

Four different naturally selected variants of TK/OR/71-delNS1_{[1-124]} (D-del-pc1, D-del-pc2, D-del-pc3 and D-del-pc4) were studied. These variants contain deletions at the C-terminus of the NS1 gene that encode amino acid residues which differ from the wild-type sequences due to a natural introduction of a frame shift and a new stop codon in the deleted NS1 protein gene. Exemplary delNS1 genes include:

| | | SEQ ID NO. of | SEQ ID NO. of |
|---|---|---|---|
| | No. of aa residues of NS1 protein: | nonconsensus | full-length |
| | 1-X . . .(Y) | sequence | sequence |
| D-del-pc2 | 1-115 . . . 125 (SLSYSINWRH) | SEQ ID NO. 1 | SEQ ID NO. 5 |
| D-del-pc1 | 1-80 . . . 90 (DISLTEGFHR) | SEQ ID NO. 3 | SEQ ID NO. 6 |
| D-del-pc3 | 1-69 . . . 86 | SEQ ID NO. 4 | SEQ ID NO. 7 |
| | (RKSQDCNCIQSCSSVHY) | | |
| D-del-pc4 | 1-91 . . . 93 (EL) | | SEQ ID NO. 8 |

SEQ ID NO. 2 (GenBank accession number:
AAB93939.1):
MDSNTITSFQVDCYLWHIRKLLSMRDMCDAPFDDRLRRDQKALKGRGSTL
GLDLRVATMEGKKIVEDILKSETDENLKIAIASSPAPRYITDMSIEEISR
EWYMLMPRQKITGGLMVKMDQAIMDKRITLKANFSVLFDQLETLVSLRAF
TDDGAIVAEISPIPSMPGHSTEDVKNAIGILIGGLEWNDNSIRASENIQR
FAWGIRDENGGPPLPPKQKRYMARRVESEV
SEQ ID NO. 5:
MDSNTITSFQVDCYLWHIRKLLSMRDMCDAPFDDRLRRDQKALKGRGSTL
GLDLRVATMEGKKIVEDILKSETDENLKIAIASSPAPRYITDMSIEEISR
EWYMLMPRQKITGGLSLSYSINWRH
SEQ ID NO. 6:
MDSNTITSFQVDCYLWHIRKLLSMRDMCDAPFDDRLRRDQKALKGRGSTL
GLDLRVATMEGKKIVEDILKSE TDENLKIADISLTEGFHR
SEQ ID NO. 7:
MDSNTITSFQVDCYLWHIRKLLSMRDMCDAPFDDRLRRDQKALKGRGSTL
GLDLRVATMEGKKIVEDILRKS QDCNCIQSCSSVHY
SEQ ID NO. 8:
MDSNTITSFQVDCYLWHIRKLLSMRDMCDAPFDDRLRRDQKALKGRGSTL
GLDLRVATMEGKKIVEDILKSETDENLKIAIASSPAPRYITEL

NS1 deletion mutants can be naturally occurring mutants or variants, or spontaneous mutants can be selected that have an impaired ability to block the induction of IFN in infected cells. In another embodiment, mutant influenza viruses that hyper produce IFN can be generated by exposing the virus to mutagens, such as UV radiation or chemical mutagens, or by multiple passages and/or passage in non-permissive hosts, or by screening in a differential growth system, or by isolation and replication of preferred particles through end-point dilution of the influenza virus quasi-species thereby screening for those mutants with enhanced IFN-inducing capacity.

In another embodiment, NS1 mutations can be engineered into a negative strand RNA virus such as influenza using “reverse genetics” approaches. In this way, natural or other mutations which confer the attenuated phenotype can be engineered into vaccine strains. For example, but not limited to, deletions, insertions or substitutions of the coding region of the gene responsible for preventing IFN induction (such as the NS1 gene of influenza) can be engineered. Deletions, substitutions or insertions in the non-coding region of the gene responsible for preventing IFN induction are also contemplated. To this end, mutations in the signals responsible for the transcription, replication, polyadenylation and/or packaging of the gene responsible for preventing/suppressing IFN induction can be appropriately engineered. For example, in influenza virus, such modifications can include but are not limited to: substitution of the non-coding regions of an influenza A virus gene by the non-coding regions of an influenza B virus gene, base pair exchanges in the non-coding regions of an influenza virus gene, mutations in the promoter region of an influenza virus gene, and substitutions and deletions in the stretch of uridine residues at the 5′ end of an influenza virus gene affecting polyadenylation. Such mutations, for example, to the promoter, could down-regulate the expression of the gene responsible for preventing IFN induction. Mutations in viral genes which may regulate the expression of the gene responsible for preventing IFN induction are also within the scope of viruses that can be employed. For example, insertion of uridines in the coding sequences of viral RNA to tailor the dose of UV radiation that is required to optimize IFN induction by the vaccine.

Some NS1 deletions may not result in an altered IFN induction phenotype, but rather in altered viral functions and an attenuated phenotype, e.g., altered inhibition of nuclear export of poly(A)-containing mRNA, altered inhibition of pre-mRNA splicing, altered inhibition of the activation of PKR by sequestering of dsRNA, altered effect on translation of viral RNA and altered inhibition of polyadenylation of host mRNA.

The reverse genetics technique includes the preparation of synthetic recombinant viral RNAs that contain the non-coding regions of the negative strand virus RNA which are essential for the recognition by viral polymerases and for packaging signals necessary to generate a mature virion. The recombinant RNAs are synthesized from a recombinant DNA template and reconstituted in vitro with purified viral polymerase complex to form recombinant ribonucleoproteins (RNPs) which can be used to transfect cells. In another embodiment, the negative strand virus RNA may be synthesized from complementary positive strand RNA.

Further, it is shown that specific sequences of nonconsensus amino acids at the C-termini of truncated NS1 proteins can increase induction/production of IFN several fold and play a role in the host cell dependence of IFP efficiency.

For example, the unusual sequence encoding the 10 amino acids at the C-terminus of D-del-pc2 (SLSYSINWRH; SEQ ID NO: 1) suffices to convert a weak inducer of IFN into a hyper-inducer in the appropriate host, along with a concomitant change in the nature of the IFN-induction dose-response curve. The naturally selected D-del-pc2 (delNS1-[1-115 . . . 125]) variant induced from 5- to 70-times more IFN in mammalian cells than the engineered variants in which (i) the 10 nonconsensus amino acid sequence was removed delNS1-[1-115], and (ii) the 10 aa nonconsensus residues were replaced with the normal sequence [1-125] (FIG. 8; FIG. 9). This demonstrates the uniqueness of this sequence for creating hyperactive IFPs in proper host species. The amount of IFN induced by the naturally selected D-del-pc2 (delNS1-[1-115 . . . 125]) variant may be more than 2-fold or more than 70-fold higher compared to that induced by delNS1-[1-115] or delNS1-[1-125] variants depending on the cells. The reversal in phenotype of the efficiency of IFPs with a change in the species of host cells as demonstrated herein, could have been predicted. The quantitative analysis of the influenza virus subpopulations, in particular the IFN-inducing capacity of these variants in different host cells demonstrated herein, allowed that distinction to be made. The proper match between variant and host species is related to the IFN inducing prowess of each variant.

In certain embodiments, the IFPs and DIPs are generated in the same virus preparation. It is noted that the delNS1 variants described herein naturally generate optimized subpopulations of IFPs and DIPs.

In certain embodiments, IFPs can be added from another source such as a population of an influenza virus with a second delNS1 gene or a population of a hyper IFN inducing wild type virus such as that isolated from the influenza virus quasispecies by randomly or systematically screening through, but not limited to, endpoint dilution.

In one embodiment, the IFPs and the DIPs are produced from a human influenza virus in which the NS gene segment is replaced with the NS gene segment from another influenza virus, preferably a H5N1 virus. Unexpectedly, this modified influenza virus displays low pathogenesis, produces large numbers of DIPs, highly efficient IFPs and does not suppress IFN induction. Without being held to theory, it is believed that there is an incompatibility between the NS1 protein expressed by the inserted NS gene segment and the remaining seven gene segments of the virus that produces the above observed advantages, or that the NS1 protein from H5N1-NS gene is not an intrinsic suppressor/inhibitor of IFN induction.

DIPs are noninfectious particles that are generated when influenza virus is grown at high multiplicities of infectious particles. Since a single DIP typically suffices to block specifically the replication of infectious virus, virologists tend to use low multiplicities of infection to prevent their formation. However, the inventors of the present application have discovered that there is a correlation between the synthesis of truncated NS1 proteins and the generation of DIPs during infection. Thus, the D-del-pc1, D-del-pc2, D-del-pc3, and D-del-pc4 variants which express delNS1 proteins with non-consensus amino acids at the C-terminus also contain large subpopulations of DIPs. Without being held to theory, it is believed that the action of DIPs enhances the efficiency of IFPs because small defective-interfering RNAs delivered by the DIPs out-compete the synthesis of a cognate viral polymerase subunit. This in turn also reduces or totally blocks production of new functional polymerase proteins/complexes of influenza virus which normally turn off IFN-induction pathways. Importantly, DIPs block the replication of influenza virus independent of their hemagglutinin (HA) subtype.

The subpopulation of DIPs can be from the same preparation as the IFPs (i.e., a preparation of an influenza virus with a delNS1 gene) and/or DIPs are added from another source.

DIPs are described in US 2009/0191158, incorporated herein by reference for its description of DIPs. In one embodiment, a DIP or DI particle is an influenza virus particle which contains a genomic RNA, usually one of the 3 polymerase gene segments, with an internal deletion, that leaves the 5′ and 3′ promoter regions and packaging signals intact, such that the virus is non-infectious and can only replicate in the presence of a “helper virus particle” (a virus particle with a completely functional genome). DI influenza viruses include, for example, the spontaneously produced DI influenza A virus (A/equine/Newmarket/7339/79 (H3N8)), and DI virus A/WSN (H1N1). Cloned DI viruses can be produced by the methods of US 2009/0191158, which includes transfecting a cell with a plasmid which produces an influenza virus RNA segment with an internal deletion. Cloned DI viruses described in US 2009/0191158 include Cloned DI Virus 220/PR8 and Cloned DI Virus 244/151PR8. In one embodiment, the subpopulation of DIPs is prepared from a defective influenza virus that contains an RNA with a deletion such that the virus is non-infectious. Some influenza viruses have a high propensity to generate DIPs and hence can be used to produce M-LAIV/H-IFPs.

In one embodiment the ratio of DIPs to IFPs is greater than 10:1 or higher as determined in mammalian or avian cells. Certain preparations have different DIP:IFP ratios in mammalian compared to avian cells. For example, D-del-pc4 has a DIP:IFP ratio of 2.4 in L(Y) or 1.8 in Marc-145 mammalian cells and 66.7 in avian cells, making it a useful preparation in avian species and not a useful preparation in mammalian species.

The vaccine preparations described herein are grown in embryonated eggs, as are most current candidate LAIVs. The vaccine preparations and populations may also be grown in mammalian or avian cell cultures. Inoculation of M-LAIV/H-IFPs into the allantoic fluid of eggs, or in appropriate cell cultures, naturally generates influenza virus particle subpopulations. The subpopulation particle composition of the M-LAIV/H-IFPs preparations is described below in more general terms.

Infectious virus particles are measured as plaque-forming particles (PFPs) or egg-infectious dose₅₀ units (EID₅₀). These represent about 1 to fewer than 10 out of every 100 hemagglutinating (physical)-particles (HAPs) in a population of influenza virus.

In addition to IFPs and DIPs, influenza virus preparations contain other particle types. Interferon induction-suppressing particles (ISPs) act to suppress the IFN inducing capacity of IFPs in cells otherwise programmed to produce IFN. The presence of ISPs in M-LAIVs/H-IFPs is not desirable and can be avoided.

Noninfectious cell-killing particles (niCKPs) are usually present in large excess over infectious particles (which also kill cells) but do not appear to affect the efficiency of IFPs or the effectiveness of M-LAIVs/H-IFPs. In general terms cell-killing particles do not appear to interfere with the biological activities of other particle subpopulations.

All of the particles described above are subsumed in the population of hemagglutinating (physical) particles (HAPs) and constitute all of the known biologically active particles (BAPs) of influenza virus. Comparing the total numbers of BAPs with the total of HAPs indicates there is still a sizeable fraction of the HAPs without known biological activity. However, it should be noted that all HAPs carry the hemagglutinin (HA) molecules/proteins that constitute the main antigen to which antibodies that neutralize influenza virus are directed.

For the purposes of the present disclosure, the most important BAPs are the IFPs and the DIPs, and the ratio of these two particles in vaccine preparations and populations. In one embodiment, the preparations include no or low levels of IFN induction suppressing particles (ISPs).

In one embodiment, the M-LAIV/H-IFPs preparations or populations are UV irradiated with a tailored dose of UV. Such treatment increases the IFN inducing efficiency of IFPs present in M-LAIV/H-IFPs. The effective UV radiation encompasses wavelengths of 230 to 270 nm, specifically about 254 nm. Previously, high doses of UV radiation have been used to inactivate any live (i.e., infectious) virus particles in a preparation such as a DI virus preparation. However, the inventors of the present application have unexpectedly found that irradiation with tailored low doses of UV maximizes the quantum yield without totally inactivating the virus preparations. The optimal UV dose to achieve the maximal (quantum) yield (QY) of IFN from UV irradiated influenza virus is generally about 500 to 2500 ergs/mm², i.e., 5 to 30 lethal hits to the virus genome, and can be readily determined for each M-LAIV/H-IFPs vaccine. A lethal hit to the genome inactivates 63 out of 100 infectious particles. The enhanced QY of IFN following treatment with 5 to 30 lethal UV hits to the virus genome is beyond that which results solely by the loss of function due to truncation of some NS1 proteins or the action of DIPs.

The QY of IFN induced by the M-LAIV/H-IFPs described herein, and the qualitative nature of the IFN induction dose-response curve is dependent on the host species, i.e., mammalian vs. avian. This unexpected observation constitutes a novel feature of the present disclosure. Thus, the compositions described herein can be tailored for a specific host species to maximize the QY of IFN. For example, variant D-delNS1-pc2 is a weak inducer of IFN in chicken cells, and an ineffective vaccine LAIV in chicken. Yet surprisingly, this same virus is a hyper-inducer of IFN in mammalian cells (monkey and mouse) and is an effective vaccine as tested in mice.

The M-LAIV/H-IFPs variants self-generate particle subpopulations that all express C-terminally truncated NS1 proteins, in which are subsumed large subpopulations of DIPs and IFPs. Both the uniquely truncated delNS1 proteins with nonconsensus amino acids at the C-terminus, and the DIPs contribute to the hyper-production of IFN in cells infected with IFPs. High multiplicity serial passages of the virus may enrich DIP production but are not necessary to produce DIPs. Further, UV (approximately 254 nm)-radiation doses tailored to each LAIV variant, further enhance the IFN-inducing capacity of the IFPs in the host cell. It is the mixture of the virus particles in the M-LAIV/H-IFPs that are generated during growth of the virus that results in hyper-induction /production of IFN by highly efficient IFPs that provides exceptional interferon-inducing ability.

Also included herein are methods of inducing interferon in an individual having or suspected of having a viral infection comprising administering compositions comprising populations of IFPs and DIPs as described herein. In one embodiment, the individual is a mammal or an avian and the ratio of DIPs to IFPs is greater than 10:1. In one embodiment, the subpopulation of IFPs contains a delNS1 gene. In another embodiment, the subpopulation of TFPs contains a full size NS1 gene.

Pharmaceutical compositions include IFPs and DIPs and a pharmaceutically acceptable carrier. A pharmaceutical composition is formulated to be compatible with its intended route of administration. Examples of routes of administration include, but are not limited to, parenteral, e.g., intravenous, intradermal, subcutaneous, oral, peroral, intranasal (e.g., inhalation), transdermal (e.g., topical), transmucosal, and rectal administration. In a specific embodiment, the composition is formulated in accordance with routine procedures as a pharmaceutical composition adapted for intravenous, subcutaneous, intramuscular, oral, intranasal, or topical administration to human beings. Typically, compositions for intravenous administration are solutions in sterile isotonic aqueous buffer.

Intranasal compositions can be formulated in an aerosol form, spray, mist or in the form of drops. One example is an aerosol spray presentation from pressurized packs or a nebulizer, with the use of a suitable propellant (e.g., dichlorodifluoromethane, trichlorofluoromethane, dichlorotetrafluoroethane, carbon dioxide or other suitable gas). In the case of a pressurized aerosol the dosage unit may be determined by providing a valve to deliver a metered amount. Capsules and cartridges (composed of, e.g., gelatin) for use in an inhaler or insufflator may be formulated containing a powder mix of the compound and a suitable powder base such as lactose or starch.

Optionally, exogenously delivered IFN, preferably perorally, can be used to enhance the adjuvant and/or antiviral effect.

The discovery of optimal ratios of DIPs to IFPs enables screening of virus preparations for the optimal ratios, and identification of virus populations that are well-suited for either mammalian or avian subjects.

In one embodiment, a method of screening an influenza virus preparation for hyper interferon-inducing capacity which exceeds by 10 fold or higher the basal levels induced by wild type influenza viruses comprises

preparing an influenza virus preparation from an influenza virus with a delNS1 gene or a full-size NS1 gene that expresses an NS1 protein lacking the capacity to suppress interferon induction, wherein the preparation contains interferon-inducing particles and defective-interfering particles, and

quantifying the ratio of defective-interfering particles to interferon-inducing particles in mammalian cells, and

determining that the influenza virus preparation is suitable for treating mammalian species when the ratio of defective-interfering particles to interferon-inducing particles in the influenza virus preparation is greater than 10:1 in mammalian cells, or

quantifying the ratio of defective-interfering particles to interferon-inducing particles in avian cells, and

determining that the influenza virus preparation is suitable for treating avian species when the ratio of defective-interfering particles to interferon-inducing particles in the influenza virus preparation is greater than 10 in avian cells.

The invention is further illustrated by the following non-limiting examples.

### EXAMPLES

### Example 1

### IFN-Inducing Capacity of Four Candidate Live-Attenuated Influenza Vaccines (LAIVs) Tested in Monkey Marc-145 and Mouse L(Y) Cells

Four candidate live-attenuated influenza vaccines (LAIVs) derived from naturally selected genetically stable variants of A/TK/OR/71-delNS1_{[1-124]} (H7N3) that differed only in the length and amino acid composition of the C terminus of the NS1 protein they encode, were distinguishable as effective (vac⁺), or ineffective (vac⁻), viruses by their ability to stimulate protective antibodies in chickens and intrinsic capacity to induce IFN in developmentally aged chicken embryonic cells (CEC) as determined by the generation and analysis of full IFN induction dose-response curves. In chicken cells, the vac⁺ variants, D-del-pc3 and D-del-pc4, induced higher quantum (maximum) yields (QYs) of IFN, relative to the vac⁻ variants, D-del-pc1 and D-del-pc2.

| | | SEQ ID NO. of |
|---|---|---|
| | No. of aa residues of NS1 protein: | nonconsensus |
| | 1-X . . .(Y) | sequence |
| D-del-pc2 | 1-115 . . . 125 (SLSYSINWRH) | SEQ ID NO. 1 |
| D-del-pc1 | 1-80 . . . 90 (DISLTEGFHR) | SEQ ID NO. 3 |
| D-del-pc3 | 1-69 . . . 86 (RKSQDCNCIQSCSSVHY) | SEQ ID NO. 4 |
| D-del-pc4 | 1-91 . . . 93 (EL) | |
| Wild type | 1-230 | |
| (Wt) | | |

The D-del-pc2 variant has a SLSYSINWRH (SEQ ID NO.1) motif at the C-terminus. The D-del-pc1 variant has a DISLTEGFHR (SEQ ID NO. 2) motif at the C-terminus. The D-del-pc3 variant has a RKSQDCNCIQSCSSVHY (SEQ ID NO. 3) motif at the C-terminus. The D-del-pc4 has an EL motif at the C-terminus. The consensus motif at the C-terminus of the wild type virus is MVKMDQAIMD (SEQ ID NO. 9).

In order to determine whether the in vitro screen of candidate LAIVs in chicken cells sufficed to predict vac⁺ candidates in mammalian hosts, the IFN-inducing capacity of these same variants was tested in monkey Marc-145 and mouse L(Y) cells; these were grown in MEM or DMEM+5% FBS and aged in culture for 9- or 4-days, respectively.

Surprisingly, there was a host cell-dependent reversal of the IFN-inducing phenotype when IFN yields from mammalian (monkey Marc-145 or mouse L(Y)) cells were compared with those from developmentally aged chicken embryonic cells (CEC) (Table 1).

TABLE 1

| | No. of aa residues | DIP:IFP ratios | | |
|---|---|---|---|---|
| rgTK/OR/71 | of NS1 protein: | | Marc-145/ | MDCK/ |
| designation† | 1-X . . . Y § | Marc-145 ∥ | L(Y) [symbol] | CEC ¶ |
| D-del-pc2 | 1-115 . . . 125 | 12.1 | 16.7 | 12.4 |
| D-del-pc1 | 1-80 . . . 90 | 16.7 | 21.6 | 6.4 |
| D-del-pc3 | 1-69 . . . 86 | 4.7 | 9.3 | 130.8 |
| D-del-pc4 | 1-91 . . . 93 | 2.4 | 1.8 | 66.7 |
| Wild type (Wt) | 1-230 | 0.73 | 5.3 | 2.9 |

| rgTK/OR/71 | Relative IFP efficiency (RIE) ^{‡} | | |
|---|---|---|---|
| designation† | Marc-145 | L(Y) | CEC |
| D-del-pc2 | 126.0 | 37.0 | 3.5 |
| D-del-pc1 | 69.0 | 2.0 | 4.3 |
| D-del-pc3 | 30.0 | 1.9 | 33.3 |
| D-del-pc4 | 6.5 | 0.2 | 20 |
| Wild type (Wt) | 1.0 | 1.0 | 1.0 |

†All delNS1 variants are in the A/TK/OR/71 influenza virus background generated using reverse genetics.
§ The (1-X) represents the N-terminal aa residues that are consensus with the wild-type A/TK/OR/71-SEPRL, and (. . . Y) represents frame shift associated residues that are not consensus with the wild-type sequences.
^{‡} The magnitude of interferon induced relative to the wild type virus.
# Measured using helper-virus-reduction assay in Marc-145 cells.
* Calculated relative to the IFN U/10⁶ IFP value for the Wt virus.
∥ DIPs and IFPs were both assayed in Marc-145 cells.
¶ DIPs were assayed in MDCK cells while IFPs were assayed in aged chick
embryonic cells. [symbol]
¶ DIPs were assayed in Marc-145 cells while IFPs were assayed in L(Y) cells.

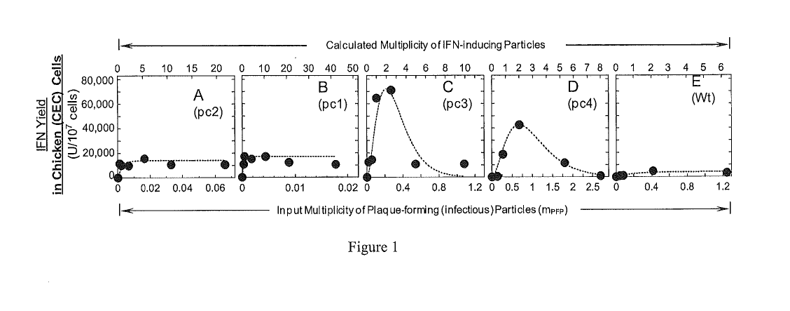

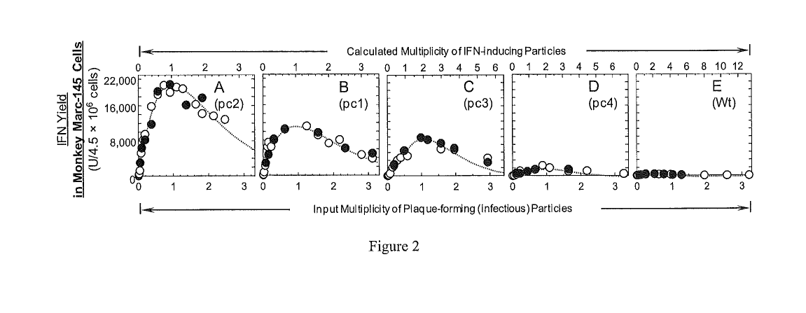

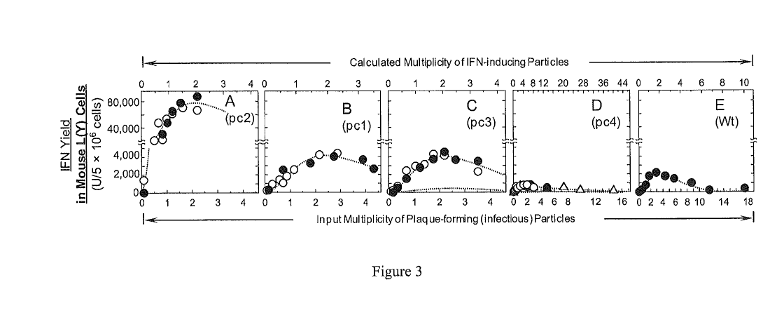

Shown below is the order of induction of increasing QYs of IFN as IFN Units per 10⁷ CEC, and approximately 5×10⁶ for both Marc-145 and L(Y) cells (FIG. 1, FIG. 2, and FIG. 3):

TABLE 2
Comparison of the order of induction of increasing QYs of IFN in different host cells

| | | | | | |
|---|---|---|---|---|---|
| CEC | Wt (4,000) | → pc2 (14,000) | → pc1 (17,000) | → pc4 (42,500) | → pc3 (72,000) |
| Marc-145 | Wt (335) | → pc4 (2,150) | → pc3 (10,000) | → pc1 (11,500) | → pc2 (21,000) |
| L(Y) | pc4 (800) | → Wt (2,130) | → pc3 (4,100) | → pc1 (4,250) | → pc2 (78,000) |

Differences in this host cell-dependent reversal of the IFN-inducing phenotype became more apparent when the relative IFN-inducing particle (IFP) efficiency (RIE) values were compared. The RIE for each variant was calculated from the titer of the IFPs, the QY of induced IFN, and the number of cells that produced the IFN, and set relative to the isogenic Wt virus which was given a value of 1.0 (Table 1). The enhanced IFN-induction capacity of delNS1 variants is thought to favor their use as LAIVs because influenza viruses are intrinsically sensitive to IFN action, and IFN acts as a potent adjuvant for stimulation of the adaptive immune responses. Both of these attributes likely contribute to the attenuation of the virus and amelioration of the disease.

The IFN-inducing capacity of delNS1 variants can be attributed to functional loss of at least: (i) NS1, and (ii) polymerase subunits; recognized as small, and large UV targets, respectively. The reversal in the IFN-inducing efficiency of IFPs in avian and mammalian cells (FIG. 1; FIG. 2; FIG. 3; Table 2) reveals a role for host-dependent factors in the regulation of IFN induction and/or production. One such factor is cell RNA polymerase II (cell pol II) which is degraded upon association with viral polymerase leading to shut off of cell mRNA transcription, including IFN-mRNA. By inactivating at least one of the 3 viral polymerase genes, the inhibition of cellular transcription would be abrogated and more IFN-mRNA would be synthesized and enhanced levels of IFN would be produced, as is observed upon low doses of UV radiation. Since most influenza virus isolates that express full-size NS1 induce some, albeit low, levels of IFN, we infer that some transcription of IFN-mRNA precedes the viral polymerase-mediated global turn-off of cell mRNA synthesis, and that its translation proceeds for some time thereafter. This seems plausible since IFN-mRNA appears in cells soon after infection. Viral polymerase also was implicated in the inhibition of IFN induction by blocking the activation of IPS1/MAVS-dependent IFN-induction pathways. Thus, viral polymerase suppresses the signaling of IFN induction, as well as IFN mRNA transcription, independently of the NS1 protein.

### Example 2

### RT-PCR Detection of Polymerase Gene Segment-Derived Subgenomic Defective-Interfering RNAs

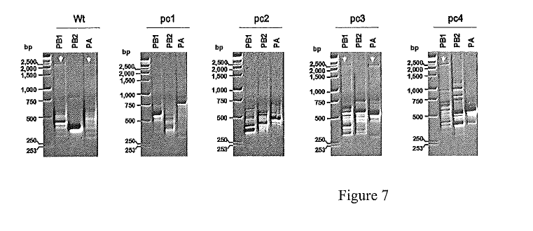

Materials and Methods: In FIG. 7, RT-PCR detection of polymerase gene segment-derived subgenomic potential defective-interfering RNAs using the following segment-specific primers: PB1, F5′-ATATAAGCAGGCAAACCATTTG-3′ (SEQ ID NO. 10), R5′-ATATCG TCTCGTATTAGTAGAAACAAGG-3′ (SEQ ID NO. 11); PB2, F5′-AGCGAAAGCAGGTCAAWTATATTCAATATG-3′ (SEQ ID NO. 12), R5′-ATATGGTCTCGTATTAGTAGAAACAAGGTCGTTT-3′ (SEQ ID NO. 13); and PA, F5′-TATTCGTCTCAGGGAGCGAAAGCAGGTAC-3′ (SEQ ID NO. 14); R5′-ATATCGTCTCGTATTAGTAGAAACAAGGTACTT-3′ (SEQ ID NO. 15). Viral RNA from egg-grown virus stocks was extracted using QIAamp® Viral RNA Mini Kit (Qiagen®). RT-PCR was carried out using OneStep RT-PCR kit (Qiagen®) under previously reported conditions. Template vRNA used in the reverse transcription step was standardized to 437 ng per reaction for all viruses. The RT, 30 min at 50° C.; PCR amplification, 35 cycles of melting at 94° C. for 45 s, annealing at 53° C. for 15 s, and primer extension at 72° C. for 150 s; final extension, 10 min. Primer specificity was determined by ability to amplify the target segments in reactions with mixed cDNAs from all the 8 segments. Furthermore, 6 cDNA bands representing DI-RNAs were randomly chosen, cloned into PCR®2.1-TOPO vector (Invitrogen) and sequenced to confirm that the primer targeted the projected segment (GenBank Accession Numbers JQ436726 for PA-(SEQ ID NO. 16), JQ436727 for PB2-(SEQ ID NO. 17), and JQ436728 for PB1-(SEQ ID NO. 18) representative of a band of defective RNA from pc4, pc3, and pc2, respectively). Arrows show cDNA bands representing full size genomic RNAs.

Based on the above results, the inventors propose a novel means to boost IFN induction by IFPs through functional inactivation of viral polymerase by the co-introduction of defective-interfering particles (DIPs) into IFP-infected cells. Without being held to theory, it is proposed that the preferential synthesis of the small defective-interfering RNAs (DI-RNAs) (FIG. 7) of DIPs significantly reduces the synthesis of viral polymerase and mRNAs, and hence functional protein, thereby preventing turn-off of cell pol II and IFN-mRNA transcription as discussed above. The inventors invoke this model to account for our observations that the most effective LAIVs contained the largest subpopulations of DIPs as measured by helper-virus-reduction assays. Importantly, DIPs of themselves do not induce IFN as inferred from the high ratios of DIP:IFP subpopulations observed in the delNS1 variants in Marc-145 cells (Table 1), and as measured in different cell types. If DIPs did function as IFPs then the IFP titers would be equal to the sum of the titers of DIP and IFP. They are not (Table 1).

The two variants, D-del-pc3 and D-del-pc4, that induced the highest amounts of IFN in CEC contained subpopulations with the highest DIP:IFP ratios relative to pc1 and pc2, the two variants that induced low amounts of IFN (Table 1). This relationship was reversed in Marc-145 cells. Variants that induced the least amount QY of IFN in aged CEC, i.e., D-del-pc1 and D-del-pc2, were scored as the best inducers in Marc-145 cells, whereas, variants that induced the highest QY of IFN in aged CEC, i.e., D-del-pc3 and D-del-pc4, were scored as the least efficient IFN inducers in Marc-145 cells (FIG. 1; FIG. 2; FIG. 3; Table 2). This reversal of the IFN-inducing phenotype with changes in the DIP:IFP ratio suggests that differences in DI-RNA sizes were not reflected in differences in IFN induction efficiency (FIG. 7). It appears that co-infection of IFP-infected cells with any size of DI-RNA suffices to enhance IFN induction. Also, it is possible that the preferential interaction of RIG-I and DI-RNAs may result in IFN induction; however, our quantitative data indicate that DIPs do not function as IFPs raising the interesting question about the significance of DI-RNA-RIG-I interaction in the context of the variants used herein.

Without being held to theory, the inventors could not attribute the differences in DI-RNA sizes to IFN inducing efficiency. Notably, DI-RNAs were synthesized from all three polymerase genes (FIG. 7). It is striking that no cDNA band was seen for the full-length PB2 segment in all four isogenic NS1 variants and Wt, suggesting that PB2-derived DI-RNAs out-compete their parental full-length RNA in replication. The predominant PB2-derived DI-RNA species in the Wt, about 350 nt long (FIG. 7), was accurately predicted by UV-target analysis. The differential synthesis of efficient DI-RNA species from PB1 or PA gene segments (FIG. 7) appears to be due to differences in the effector domains of the NS1 proteins expressed by the four delNS1 variants and Wt, with the former displaying a broader spectrum of DI-RNA sizes than the latter. Taken together, our data indicate that NS1 has a direct influence on the propensity to generate DIPs. This corroborates the role of NS1 in transcription/replication of viral RNA, independent of its function to prevent IFN induction.

In further support of our model we note that the UV dose (calculated to be approximately 2,500 ergs/mm², i.e., approximately 13 lethal hits to infectivity) inactivated virtually all residual infectivity in the DIP preparations used to treat mice, and had no effect on the activity of DIPs because of their small (approximately 400 nts) UV-target. However, this dose of UV radiation was in the range used to enhance the IFN-inducing capacity of most isolates of influenza virus, including DIP-free stocks. This model also accounts for the IFN found in mice that received large numbers of DIPs irradiated with low doses of UV, if one assumes the preparation also contained IFPs.

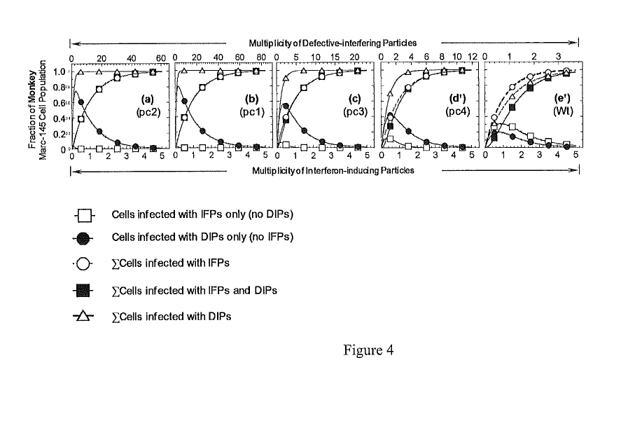

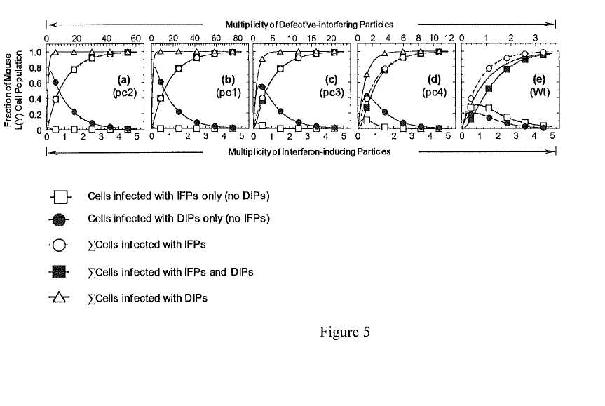

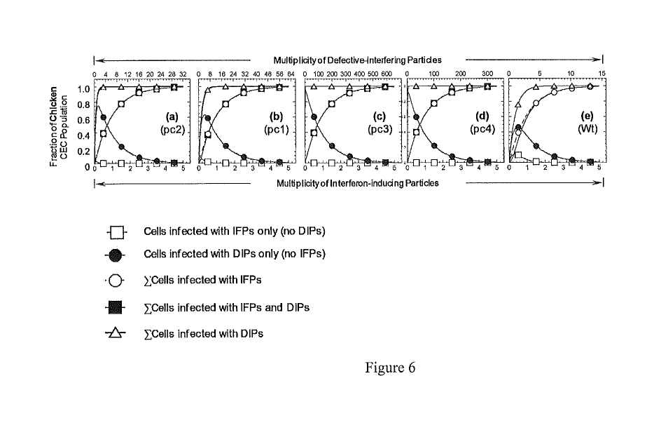

Considering the central role that IFN induction plays in the expression of the vac⁺ phenotype, and the new role proposed for DIPs, we examined the dynamics of infection by IFPs and DIPs based on a Poisson distribution of these particles in the cell population. A Perl program (subpopulon) was developed to simulate infection at each multiplicity as follows: (i) an array of 10,000 virtual cells was created; (ii) based on the observed titers (Table 1), the number of IFPs and DIPs infecting each cell was determined using the random Poisson function from the CPAN Math::Random module (Math-Random-0.70), and;(iii) cells that received either IFPs or DIPs only, or both classes of particles, were counted, expressed as a fraction of the whole population (n=10,000) and plotted as shown in FIGS. 4, 5, and 6. The fraction of the cell population infected with ≧1 particles of each class for each multiplicity is described by the probability P of having: DIPs in cells that may or may not be infected with IFPs, P_{(r≧1 DIP)}=(1−e^{-m}^{_{DIP}}); IFPs in cells that may or may not be infected with DIPs, P_{(r≧1 IFP)}=(1−e^{-m}^{_{IFP}}); DIPs together with IFPs, P_{(r≧1 DIP)}·P_{(r≧1 IFP)}; DIPs only, P_{(r≧1 DIP)}−(P_{(r≧1 DIP)}·P_{(r≧1 IFP)}); IFPs only, P_{(r≧1 IFP)}−(P_{(r≧1 DIP)}·P_{(r≧1 IFP)}), where r is the actual number of DIPs or IFPs that enter the cell, e is the base of the natural logarithm and m_{DIP} and m_{IFP} are the multiplicities of DIPs and IFPs, respectively. Several features are worthy of note in characterizing the best inducers of IFN (pc2 and pc1 in this case), and predicting vac variants (FIGS. 4; FIG. 5; FIG. 6): (1) Since the DIP content, relative to IFP, was highest for variants pc2 and pc1, the cumulative fraction of cells infected with DIPs rapidly reached 1.0 with increasing multiplicities, while those of pc3 and pc4 were slower in doing so, with Wt being the slowest. (2) The fraction of cells infected only with DIPs peaked much earlier than that infected with IFPs, supporting our contention that DIPs per se did not induce IFN. (3) The fraction of cells co-infected with IFPs and DIPs was congruent with the fraction of cells infected with IFPs for pc2 and pc1 because virtually all cells were infected with DIPs. (4) The cumulative fraction of cells infected with IFPs only (red) was highest in Wt populations because the ratio of DIP:IFP was the lowest and fewer cells were co-infected with DIPs. Thus, cells infected only with IFPs produced lower yields of IFN than those co-infected with IFPs and DIPs.

The identification and quantification of subpopulations of noninfectious biologically active particles such as DIPs, IFPs, IFN induction-suppressing particles and cell-killing particles in populations of influenza virus has stimulated interest regarding their potential function in LAIVs and role in the regulation of pathogenesis and disease. From the quantification, interaction, and analysis of IFPs and DIPs in this study we concluded that the most effective candidate LAIVs or broad spectrum antiviral reagents made from influenza viruses contain IFPs that intrinsically are maximally efficient in inducing IFN, along with DIPs that contribute further to the enhancement of IFN-induction. LAIVs modified in this manner are termed M-LAIVs. It also is possible, though not proven, that IFP-cell interactions that result in down-regulation of IFN at high multiplicities (FIG. 1; FIG. 2; FIG. 3) provide a degree of fine-tuning to virus replication that helps maximize M-LAIV efficacy and action against a broad spectrum of viruses sensitive to IFN. Thus, three conditions are proposed to help maximize the efficiency of IFPs in M-LAIV preparations: (1) truncation of NS1 to abrogate NS1-mediated suppression of IFN induction; (2) co-infection of IFP-infected cells with DIPs to compromise viral polymerase-mediated suppression of IFN-mRNA transcription through turn-off of cell pol II; (3) UV irradiation to abrogate both viral polymerase- and NS1-mediated suppression. Furthermore, the adjuvant effects of endogenously-induced IFN from IFPs that stimulate the response of the adaptive immune system might be enhanced further by the exogenous administration of IFN.

### Example 3

### Effect of the SLSYSINWRH Amino Acid Motif at the C-terminus of NS1 Protein of Variant D-del-pc2 on Interferon Inducing Capacity Tested in Mouse and Monkey Cells

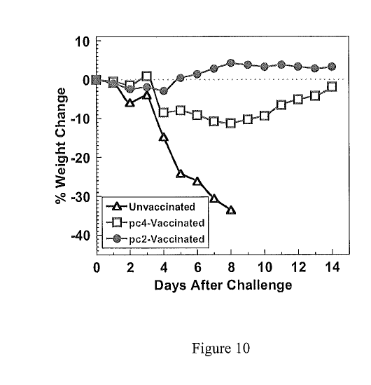

FIGS. 8 and 9 show the effect of the SLSYSINWRH (SEQ ID NO. 1) amino acid motif at the C-terminus of NS1 protein of variant D-del-pc2 on interferon inducing capacity. The ordinates represent IFN yield following exposure of the cells to increasing multiplicities of plaque-forming (infectious) particles (PFPs) (lower abscissae). The naturally selected D-del-pc2 M-LAIV/H-IFPs candidate encodes a truncated NS1 protein with a motif of 10 amino acids (aa 116-125) at the C-terminal end that are nonconsensus with those of the wildtype protein. The nonconsensus SLSYSINWRH motif (NcM) of D-del-pc2 plays a critical role in regulating defective-interfering particle to interferon-inducing particle ratios and the interferon-inducing efficiency in mammalian (mouse and monkey) cells (FIG. 8A; FIG. 9A), thereby upregulating the effectiveness of this virus as a candidate LAIV (see FIG. 10, Table 1, Table 2). Two engineered mutants with the entire SLSYSINWRH deleted (DM) (FIG. 8B; FIG. 9B) and SLSYSINWRH motif replaced with the consensus MVKMDQAIMD (SEQ ID NO: 9) motif (CM) of the wildtype virus (FIG. 8C; FIG. 9C) induced from 5 to 70 times less interferon compared to the naturally selected pc2-NcM M-LAIV/H-IFPs candidate. Note: Induction of high amounts of interferon correlated with high defective-interfering particle to interferon-inducing particle ratios (compare lower and upper abscissae).

### Example 4

### In vivo Establishment of Naturally Selected D-del-pc2-NcM as Effective M-LAIV/H-IFPs in Mice

On the basis of the reversal of IFN-inducing capacity in mammalian cells relative to chicken cells [compare FIG. 1, FIG. 2, and FIG. 3; Table 1 and Table 2] it was predicted that D-del-pc2-NcM would function as an effective M-LAIV/H-IFPs in mammals. The effectiveness of D-del-pc2-NcM (ineffective M-LAIV/H-IFPs in chickens) was compared with D-del-pc4 (effective M-LAIV/H-IFPs in chickens) in mice. D-del-pc2-NcM-vaccinated mice had a stronger initial antibody response (Table 3) which protected against weight loss (FIG. 4) and replication of the heterologous challenge virus (A/CK/NJ/02 [H7N2]) (Table 4) better than D-del-pc4-vaccinated mice.

TABLE 3
Log₂ H1 antibody titers after vaccinations and challenge

| | Control | pc2 | pc4 |
|---|---|---|---|
| 2 weeks after vaccination | 0.0 ± 0.0 | 3.7 ± 0.4 | 2.5 ± 0.6 |
| | (0/5) | (5/5) | (4/5) |
| 2 weeks after challenge | NA | 4.9 ± 0.9 | 5.1 ± 0.6 |
| | | (0.4) | (5/5) |

TABLE 4
Log₂ H1 antibody titers at 3 weeks post-vaccinations and virus titers
in nasal washes and lung homogenates 3 days after challenge

| | | | 3 days post-challenge- virus detection | | | | | | |
|---|---|---|---|---|---|---|---|---|---|
| 3 weeks post vaccination | | | | Control | | pc2 | | pc4 | |
| Log₂ H1 titer | | | | RRT- | Tissue | RRT- | Tissue | RRT- | Tissue |
| Control | pc2 | pc4 | sample | PCR | culture | PCR | culture | PCR | culture |
| 0.0 ± 0.0 | 6.7 ± 0.9 | 7.5 ± 0.7 | Nasal | 4.4 ± 0.1 | 3.1 ± 0.2 | 1.1 ± 0.3^{a,c} | 0.0 ± 0.^{0b,b} | 2.8 ± 1.6^{c,c} | 2.4 ± 0.2^{a,b} |
| (0/5) | (5/5) | (5/5) | wash | (5/5) | (5/5) | (3/5) | (0/5) | (3/5) | (2/5) |
| | | | Lung | 3.2 ± 1.1 | 4.0 ± 0.4 | 0.8 ± 0.1^{1b,b} | 2.0 ± 0.^{3b,b} | 3.5 ± 0.3^{b,b} | 2.9 ± 0.1^{a,a} |
| | | | homog. | (5/5) | (5/5) | (2/5) | (2/5) | (3/5) | (3/5) |

### Example 5

FIG. 5 shows the effect of UV (254 nm) radiation on interferon inducing capacity of M-LAIV/H-IFPs candidates. Aliquots of virus preparations that induced maximal amounts of interferon were exposed to increasing doses of UV radiation. Tailored doses of UV (254 nm) radiation can be used to enhance interferon inducing capacity of M-LAIV/H-IFPs candidates.

The use of the terms “a” and “an” and “the” and similar referents (especially in the context of the following claims) are to be construed to cover both the singular and the plural, unless otherwise indicated herein or clearly contradicted by context. The terms first, second etc. as used herein are not meant to denote any particular ordering, but simply for convenience to denote a plurality of, for example, layers. The terms “comprising”, “having”, “including”, and “containing” are to be construed as open-ended terms (i.e., meaning “including, but not limited to”) unless otherwise noted. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

While the invention has been described with reference to a preferred embodiment, it will be understood by those skilled in the art that various changes may be made and equivalents may be substituted for elements thereof without departing from the scope of the invention. In addition, many modifications may be made to adapt a particular situation or material to the teachings of the invention without departing from essential scope thereof. Therefore, it is intended that the invention not be limited to the particular embodiment disclosed as the best mode contemplated for carrying out this invention, but that the invention will include all embodiments falling within the scope of the appended claims.

1. A live attenuated influenza virus composition for inducing interferon, comprising
a subpopulation of interferon inducing particles that contain a delNS1 gene, and
a subpopulation of defective-interfering particles,
wherein the ratio of defective-interfering particles to interferon-inducing particles is greater than 10:1,
wherein the subpopulation of interferon-inducing particles, the subpopulation of defective-interfering particles, or both, is UV irradiated at 254±20 nm, at 500 to 2,500 ergs/mm², and
wherein the live attenuated influenza virus composition has hyper interferon-inducing capacity which exceeds by 10 fold or higher the basal levels induced by wild type influenza viruses.

2. The composition of claim 1, wherein the subpopulation of interferon-inducing particles and the subpopulation of defective-interfering particles are isolated from the same virus population and are irradiated together.

3. The composition of claim 1, wherein the subpopulation of defective-interfering particles are isolated and UV-irradiated followed by mixing with the subpopulation of interferon-inducing particles.

4. The composition of claim 1, wherein the subpopulation of defective-interfering particles is prepared from a defective influenza virus that contains an RNA with a deletion such that the virus is non-infectious.

5. The composition of claim 1, wherein the influenza virus is an avian or mammalian influenza virus.

6. The composition of claim 5, wherein the influenza virus is an avian influenza virus with a delNS1 gene encoding a delNS1 protein is selected from SEQ IDMO. 5, SEQ ID NO. 6, SEQ ID NO. 7, and SEQ ID NO. 8.

7. A method of inducing interferon in an individual having or suspected of having a viral infection, comprising administering to the individual a live attenuated influenza virus composition comprising
a subpopulation of interferon inducing particles that contain a delNS1 gene, or a subpopulation of interferon inducing particles that contain a full size NS1 gene, and
a subpopulation of defective-interfering particles,
wherein the ratio of defective-interfering particles to interferon-inducing particles is greater than 10, and
wherein the subpopulation of interferon-inducing particles, the subpopulation of defective-interfering particles, or both, is UV irradiated at 254±20 nm and at 500 to 2,500 ergs/mm².

8. The method of claim 7, wherein the subpopulation of interferon-inducing particles and the subpopulation of defective-interfering particles are isolated from the same viral stock or population and are UV irradiated at 254±20 nm and at 500 to 2,500 ergs/mm²together.

9. The method of claim 7, wherein the subpopulation of defective-interfering particles are isolated and UV irradiated at 254±20 nm and at 500 to 2,500 ergs/mm² followed by mixing with the subpopulation of interferon-inducing particles.

10. The method of claim 7, wherein the subpopulation of defective-interfering particles are prepared from a defective influenza virus that contains an RNA with a deletion such that the virus is non-infectious.

11. The method of claim 7, wherein the influenza virus is an avian or mammalian influenza virus.

12. The method of claim 7, wherein the individual is a mammal and the ratio of defective-interfering particles to interferon-inducing particles is greater than 10:1 in mammalian cells infected with the live attenuated influenza virus.

13. The method of claim 7, wherein the individual is a chicken and the ratio of defective-interfering particles to interferon-inducing particles is greater than 10 1 in avian cells infected with the live attenuated influenza virus.

14. The method of claim 7, wherein administration is intranasal administration.

15. The method of claim 7, wherein administration is independent of influenza virus subtype.

16. The method of claim 7, further comprising peroral administration of interferon.

17. A method of screening an influenza virus preparation for hyper interferon-inducing capacity which exceeds by 10 fold or higher the basal levels induced by wild type influenza viruses, comprising
preparing an influenza virus preparation from an influenza virus with a delNS1 gene or a full-size NS1 gene that expresses an NS1 protein lacking the capacity to suppress interferon induction, wherein the preparation contains interferon-inducing particles and defective-interfering particles, and
quantifying the ratio of defective-interfering particles to interferon-inducing particles in mammalian cells, and
determining that the influenza virus preparation is suitable for treating mammalian species when the ratio of defective-interfering particles to interferon-inducing particles in the influenza virus preparation is greater than 10:1 in mammalian cells, or
quantifying the ratio of defective-interfering particles to interferon-inducing particles in avian cells, and
determining that the influenza virus preparation is suitable for treating avian species when the ratio of defective-interfering particles to interferon-inducing particles in the influenza virus preparation is greater than 10 in avian cells.

18. The method of claim 17, wherein the influenza virus preparation is from an influenza virus with a delNS1 gene.


SEQUENCE LISTING (18 sequences)

SEQ ID NO: 1 - protein, 10 aa, Artificial Sequence
  NS1 Nonconsensus sequence
SLSYSINWRH

SEQ ID NO: 2 - protein, 230 aa, Influenza virus
MDSNTITSFQVDCYLWHIRKLLSMRDMCDAPFDDRLRRDQKALKGRGSTLGLDLRVATMEGKKIVEDILKSETDENLKIAIASSPAPRYITDMSIEEISREWYMLMPRQKITGGLMVKMDQAIMDKRITLKANFSVLFDQLETLVSLRAFTDDGAIVAEISPIPSMPGHSTEDVKNAIGILIGGLEWNDNSIRASENIQRFAWGIRDENGGPPLPPKQKRYMARRVESEV

SEQ ID NO: 3 - protein, 10 aa, Artificial Sequence
  NS1 nonconsensus sequence
DISLTEGFHR

SEQ ID NO: 4 - protein, 17 aa, Artificial Sequence
  NS1 nonconsensus sequence
RKSQDCNCIQSCSSVHY

SEQ ID NO: 5 - protein, 125 aa, Artificial Sequence
  Deleted NS1 pc2 sequence
MDSNTITSFQVDCYLWHIRKLLSMRDMCDAPFDDRLRRDQKALKGRGSTLGLDLRVATMEGKKIVEDILKSETDENLKIAIASSPAPRYITDMSIEEISREWYMLMPRQKITGGLSLSYSINWRH

SEQ ID NO: 6 - protein, 90 aa, Artificial Sequence
  Delteted NS1 pc1 sequence
MDSNTITSFQVDCYLWHIRKLLSMRDMCDAPFDDRLRRDQKALKGRGSTLGLDLRVATMEGKKIVEDILKSETDENLKIADISLTEGFHR

SEQ ID NO: 7 - protein, 86 aa, Artificial Sequence
  Deleted NS1 pc3 sequence
MDSNTITSFQVDCYLWHIRKLLSMRDMCDAPFDDRLRRDQKALKGRGSTLGLDLRVATMEGKKIVEDILRKSQDCNCIQSCSSVHY

SEQ ID NO: 8 - protein, 93 aa, Artificial Sequence
  Deleted NS1 pc4 sequence
MDSNTITSFQVDCYLWHIRKLLSMRDMCDAPFDDRLRRDQKALKGRGSTLGLDLRVATMEGKKIVEDILKSETDENLKIAIASSPAPRYITEL

SEQ ID NO: 9 - protein, 10 aa, Artificial Sequence
  wild type NS1 C-terminus
MVKMDQAIMD

SEQ ID NO: 10 - DNA, 22 bp, Artificial Sequence
  Primer
atataagcaggcaaaccatttg

SEQ ID NO: 11 - DNA, 28 bp, Artificial Sequence
  Primer
atatcgtctcgtattagtagaaacaagg

SEQ ID NO: 12 - DNA, 30 bp, Artificial Sequence
  Primer
agcgaaagcaggtcaawtatattcaatatg

SEQ ID NO: 13 - DNA, 34 bp, Artificial Sequence
  Primer
atatggtctcgtattagtagaaacaaggtcgttt

SEQ ID NO: 14 - DNA, 29 bp, Artificial Sequence
  Primer
tattcgtctcagggagcgaaagcaggtac

SEQ ID NO: 15 - DNA, 33 bp, Artificial Sequence
  Primer
atatcgtctcgtattagtagaaacaaggtactt

SEQ ID NO: 16 - DNA, 514 bp, Artificial Sequence
  clone D-del pc4 defective subgenomic RNA gene
tttgtgcgacaatgcttcaatccatgatcgtcgagcttgcggaaaaagcaatgaaagaatatggagaggatccaaaaatcgaaacaaacaaattcgctgcgatatgtacacatctggaagtgtgtttcatgtattcagatttccacttcattgatgaacgaggtgaatcgataattgtagagtctggtgatccaaatgcactcttggaaaggtctgcaggacattactagcaaagtctgtattcaacagtttgtatgcatccccacaacttgagggattttcagctgagtcaaggaagctgcttcttattgttcaggcacttagggacaacctggaacctggtactttcgatcttgggggctatatgaagcaattgaggagtgcctgattaatgatccctgggttttgcttaatgcatcttggttcaactccttccttacacatgcactgaaatagctgtggcaatgctactatttgctatccatattgtccaaaaaagtaccttgtttctact

SEQ ID NO: 17 - DNA, 543 bp, Artificial Sequence
  clone D-del pc3 defective subgenomic RNA gene
aaggtgtcatccgatttactgttcttgaagtcccagcgcaaaatgccataagtaccacgttcccttatactggagatcctccatacagccatggaacaggaacaggatacaccatggacacggtcaacagaacacatcaatattcagaaaaggggaagtggacaacaaacacggaaactggagcgccccagcttaacccaattgttgcaactacacactcctggattcctaagaggaaccgctctatcctcaacacaagccaaaggggaattcttgaggatgaaaagatgtatcaaaagtgctgcaacctgttcgagaaattcttccccagtagttcatacagaaggccggttggaatctctagcatggtggaggccatggtgtctagggcccggattgatgccaggattgacttcgagtctgggcggattaagaaagaagagttcgctgagatcatgaagatctgttccaccattgaagagctcagacggcaaaaatagtgaatttagcttgtccttcatgaaaaaatgccttgtttctact

SEQ ID NO: 18 - DNA, 313 bp, Artificial Sequence
  clone D-del pc2 defective subgenomic RNA gene
tatggccgtaatcaaaaagtacacatcagggagacgagagaagaaccccgcactcaggatgaagtggatgatggcaatgaaatatccaattacggcagataagaggataatggcccagcattgagcatcaacgaactgagcaatcttgcaaaaggagagaaggctaatgtgctaattgggcaaggagacgttgtgttggtaatgaaacggaaacgggactctagcatacttactgacagccagacagcgaccaaaagaattcggatggccatcaattagtgtcgaattgtttaaaaacgaccttgtttctact